(19) United States
(12) Patent Application Publication
(10) Pub. No.: US 2017/0044577 A1
(43) Pub. Date: Feb. 16, 2017

(54) Integrated Cellulosic Ethanol Production Process

(71) Applicant: ENCHI CORPORATION, Wellesley Hills, MA (US)

(72) Inventors: Zachary LOSORDO, Wellesley Hills, MA (US); Justin VAN ROOYEN, Wellesley Hills, MA (US); John MCBRIDE, Wellesley Hills, MA (US); Alan BELCHER, Wellesley Hills, MA (US)

(21) Appl. No.: 15/305,819

(22) PCT Filed: Apr. 20, 2015
(86) PCT No.: PCT/US15/26692

§ 371 Date: Oct. 21, 2016

Related U.S. Application Data
(60) Provisional application No. 61/982,642, filed on Apr. 22, 2014.

(51) Int. Cl. C12P 7/10

(52) U.S. Cl. CPC C12P 7/10

(57) ABSTRACT

Integrated cellulosic ethanol and corn ethanol production processes reduce the capital and operating costs of cellulosic ethanol production through high levels of integration with pre-existing corn ethanol processing equipment. The processes comprise separating corn starch from other, non-fermentable corn components (e.g. germ, protein, fiber, etc.) and cofermenting sugars derived from the corn starch in the presence of a pretreated cellulose feed. The cofermentation can be carried out using one or more hemicellulose sugar utilizing yeast strains, for example, such as one or more yeast strains.

### BACKGROUND OF THE INVENTION

Ethanol production from corn is the primary method of ethanol production in the United States. In 2013, there were 211 corn ethanol facilities in the U.S. capable of producing a total of about 14.8 billion gallons of ethanol annually. See, e.g., Battling for the Barrel: 2013 Ethanol Industry Outlook. Washington, D.C.: Renewable Fuels Association, 2013, the entirety of which is incorporated herein by reference.

The most common process to convert corn to ethanol is the dry grind process. The dry grind process involves grinding corn into corn flour, slurrying the flour with water, treating the resulting slurry, also called a mash, with heat and enzyme to liquefy starch in the mash, followed by hydrolysis and fermentation of the resulting sugars to produce ethanol. Ethanol is recovered via distillation, leaving a residue of high protein material referred to as “distiller's dried grains with solubles,” which is commonly sold as animal feed. See, generally, “A Guide to Distiller's Dried Grains with Solubles (DDGS),” 3rd Ed., U.S. Grains Council, published September 2012, the entirety of which is incorporated herein by reference. A block diagram of a standard corn ethanol production process is shown in FIG. 1.

Sales of DDGS contribute significantly to the profitability of corn ethanol production facilities. As a result, changes to corn ethanol production processes need to be closely monitored to ensure that these changes do not affect the quality of the DDGS for subsequent resale. For example, although it would be desirable to integrate a cellulosic ethanol process into a corn ethanol dry grind plant, the amount of potential integration is limited because of impurities, such as lignin, that are introduced into the DDGS via an integrated process. In addition to diluting out protein and fat in the DDGS, lignin is not digestible by ruminants and may render the DDGS unpalatable for animals. Lignin also discolors the DDGS, potentially affecting its resale value.

It would therefore be desirable to develop a process that integrates cellulosic ethanol production and corn ethanol production, while eliminating the potential for DDGS contamination.

### BRIEF SUMMARY OF THE INVENTION

The present disclosure provides integrated cellulosic ethanol and corn ethanol production processes. The disclosed processes reduce the capital and operating costs of cellulosic ethanol production through high levels of integration with pre-existing corn ethanol processing equipment. More specifically, the processes disclosed herein comprise separating corn starch from other, non-fermentable corn components (e.g. germ, protein, fiber, etc.) and cofermenting sugars derived from the corn starch in the presence of a pretreated cellulose feed. The cofermentation can be carried out using one or more hemicellulose sugar utilizing yeast strains, for example, such as one or more yeast strains disclosed in U.S. Pat. No. 8,470,592; U.S. Pat. No. 8,658,398; U.S. Ser. No. 14/188,360; U.S. Ser. No. 13/130,549; U.S. Ser. No. 13/141,952; U.S. Ser. No. 13/514,519; U.S. Ser. No. 13/459,804; U.S. Ser. No. 13/640,223; U.S. Ser. No. 13/391,554; U.S. Ser. No. 13/696,207; U.S. Ser. No. 13/814,616; U.S. Ser. No. 14/110,075; U.S. Ser. No. 14/075,846; U.S. 61/866,338; U.S. Ser. No. 12/677,428; U.S. Ser. No. 12/992,001; U.S. Ser. No. 12/599,425; U.S. Ser. No. 12/992,003; U.S. Ser. No. 13/124,255; U.S. Ser. No. 13/201,257; U.S. Ser. No. 13/393,093; PCT/US2011/039192; PCT/US2011/066968; PCT/US2012/064457; PCT/US2012/057952; PCT/US2012/067216; PCT/US2013/070964; PCT/US2013/000090, PCT/US2014/025460; and/or US 2013/0323822, each of which is incorporated herein by reference in its entirety.

Unlike a standard corn ethanol production process in which the germ and fiber are exposed to fermentation conditions, the embodiments of the integrated processes described herein can separate edible corn components from the fermentable materials, allowing the edible corn components (either as individual components or as a mixture) to be sold as feed supply without any concerns that these materials will contain inedible/unpalatable lignocellulose derived byproducts.

In on embodiment, the present disclosure provides a corn-ethanol production method comprising fractionating corn to produce corn starch; converting the corn starch into maltodextrin; and cofermenting hemicellulose sugars and the maltodextrin in a fermentation reactor in the presence of a yeast capable of fermenting hemicellulose sugar and glucose to ethanol; wherein the yeast is further capable of converting the maltodextrin to glucose or wherein the maltodextrin is treated with a glucoamylase prior to cofermentation.

In some embodiments, the fractionating is wet fractionating.

In some embodiments, the wet fractionating comprises steeping, degermination, milling, fiber separation, and protein separation.

In some embodiments, the steeping comprises soaking the corn in an aqueous solution optionally including SO₂ or an enzyme.

In certain embodiments, converting the corn starch into maltodextrin comprises liquefaction.

In some embodiments, the liquefaction comprises cooking the corn starch in the presence of a heat-stable enzyme at a temperature above about 100° C. in the presence of a shearing force.

In some embodiments, the heat-stable enzyme is an α-amylase.

In some embodiments, the hemicellulose sugars are derived from at least a first pretreatment of a cellulosic feed optionally comprising corn fiber.

In some embodiments, the at least a first pretreatment has a severity of from about 3.7 to about 4.1.

In some embodiments, maltodextrin is treated with a glucoamylase prior to fermentation.

In some embodiments, before being fed to the fermentation reactor, the hemicellulose sugars and the maltodextrin comprise a fermentation feed having a total sugar concentration of about 100 g/L to about 500 g/L.

In certain embodiments, the total sugar concentration is about 250 g/L to about 350 g/L.

In still other embodiments, the total sugar concentration is about 300 g/L.

In some embodiments, maltodextrin comprises from about 50% to about 90% by weight of the total sugar concentration in the fermentation feed.

In other embodiments, the maltodextrin comprises about 83% by weight of the total sugar concentration in the fermentation feed.

In certain embodiments, the fractionating comprises dry fractionating the corn to separate fiber and germ from endosperm.

In certain embodiments, the dry fractionating comprises milling.

In certain embodiments, the fractionating further comprises wet fractionating the endosperm to separate corn starch and protein present in the endosperm from each other.

In certain embodiments, the wet fractionating comprises steeping.

In certain embodiments, the steeping comprises soaking the corn in an aqueous solution optionally including SO₂ or an enzyme.

In certain embodiments, converting the corn starch into maltodextrin comprises liquefaction.

In certain embodiments, the liquefaction comprises cooking the corn starch in the presence of a heat-stable enzyme at a temperature above about 100° C. in the presence of a shearing force.

In certain embodiments, the heat-stable enzyme is an α-amylase.

In certain embodiments, the hemicellulose sugars are derived from the pretreatment of a cellulosic feed optionally comprising corn fiber.

In certain embodiments, the pretreatment has a severity of from about 3.7 to about 4.1.

In certain embodiments, the maltodextrin is treated with a glucoamylase prior to cofermentation.

In certain embodiments, before being fed to fermentation reactor, the hemicellulose sugars and the maltodextrin comprise a fermentation feed having a total sugar concentration of about 100 g/L to about 500 g/L.

In certain embodiments, the total sugar concentration is about 250 g/L to about 350 g/L.

In certain embodiments, the total sugar concentration is about 300 g/L.

In certain embodiments, maltodextrin comprises from about 50% to about 90% by weight of the total sugar concentration in the fermentation feed.

In certain embodiments, the maltodextrin comprises about 83% by weight of the total sugar concentration in the fermentation feed.

In another embodiment, the present disclosure provides a corn-ethanol production method comprising fractionating corn to produce a mixture of protein and corn starch; converting the corn starch in the mixture of protein and corn starch to maltodextrin in the presence of the protein; separating the maltodextrin from the protein; and cofermenting hemicellulose sugars and the maltodextrin in a fermentation reactor in the presence of a yeast capable of fermenting hemicellulose sugar and glucose to ethanol wherein the yeast is further capable of converting the maltodextrin to glucose or wherein the maltodextrin is treated with a glucoamylase prior to cofermentation.

In certain embodiments, converting the corn starch in the mixture of protein and corn starch to maltodextrin in the presence of the protein comprises liquefaction of the mixture of protein and corn starch.

In certain embodiments, the liquefaction comprises cooking the corn starch and protein in the presence of a heat-stable enzyme at a temperature above about 100° C. in the presence of a shearing force.

In certain embodiments, the heat-stable enzyme is an α-amylase.

In certain embodiments, the hemicellulose sugars are derived from the pretreatment of a cellulosic feed optionally comprising corn fiber.

In certain embodiments, the pretreatment has a severity of from about 3.7 to about 4.1.

In certain embodiments, the maltodextrin is treated with a glucoamylase prior to cofermentation.

In certain embodiments, before being fed to the fermentation reactor, the hemicellulose sugars and the maltodextrin comprise a fermentation feed having a total sugar concentration of about 100 g/L to about 500 g/L.

In certain embodiments, the total sugar concentration is about 250 g/L to about 350 g/L.

In certain embodiments, the total sugar concentration is about 300 g/L.

In certain embodiments, the maltodextrin comprises from about 50% to about 90% by weight of the total sugar concentration in the fermentation feed.

In certain embodiments, the maltodextrin comprises about 83% by weight of the total sugar concentration in the fermentation feed.

In certain embodiments, separating the maltodextrin and protein comprises centrifuging the protein away from the maltodextrin.

In certain embodiments, the centrifuging comprises using a hydrocyclone.

In another embodiment, the present disclosure provides an ethanol processing plant comprising a steeping stage; a degermination stage; a milling stage; a fiber separation stage; a protein separation stage; a liquefaction stage; and a fermentation stage; wherein the degermination stage separates starch, protein, and fiber from germ; the fiber separation stage separates fiber from the starch and protein; the protein separation stage separates the protein from the starch; and the fermentation stage is adapted to receive a fermentation feed comprising maltodextrin produced in the liquefaction stage and C5-enriched materials produced during pretreatment of a cellulosic feed supply.

In another embodiment, the present disclosure provides an ethanol processing plant comprising a tempering stage; a milling stage; a fiber and germ separation stage; a steeping stage; a protein separation stage; a liquefaction stage; and a fermentation stage; wherein the tempering stage dries corn to a selected water content; the fiber and germ separation stage separates fiber and germ from each other and from endosperm; the steeping stage steeps the endosperm isolated during the fiber and germ separation stage to produce a steeped endosperm; the protein separation stage separates protein from starch in the steeped endosperm; and the fermentation stage is adapted to receive a fermentation feed comprising maltodextrin produced in the liquefaction stage and C5-enriched materials produced during pretreatment of a cellulosic feed supply.

In another embodiment, the present disclosure provides an ethanol processing plant comprising a tempering stage; a milling stage; a fiber and germ separation stage; a liquefaction stage; a protein separation stage; and a fermentation stage; wherein the tempering stage dries corn to a selected water content; the fiber and germ separation stage separates fiber and germ from each other and from endosperm; the liquefaction stage reduces starch in the endosperm to maltodextrin; the protein separation stage separates protein from maltodextrin produced during the liquefaction stage; and the fermentation stage is adapted to receive a fermentation feed comprising the maltodextrin and C5-enriched materials produced during pretreatment of a cellulosic feed supply.

In certain embodiments, edible corn components are removed, isolated, and/or recovered during the fractionating.

In certain embodiments, edible corn components are removed, isolated, and/or recovered prior to cofermenting.

### BRIEF DESCRIPTION OF THE DRAWINGS/FIGURES

The foregoing summary, as well as the following detailed description of the embodiments, will be better understood when read in conjunction with the appended figures. For the purpose of illustration, the figures may describe the use of specific embodiments. It should be understood, however, that the integrated processes described herein are not limited to the precise embodiments discussed or described in the figures.

FIG. 1 is a diagram of a standard corn ethanol dry grind process.

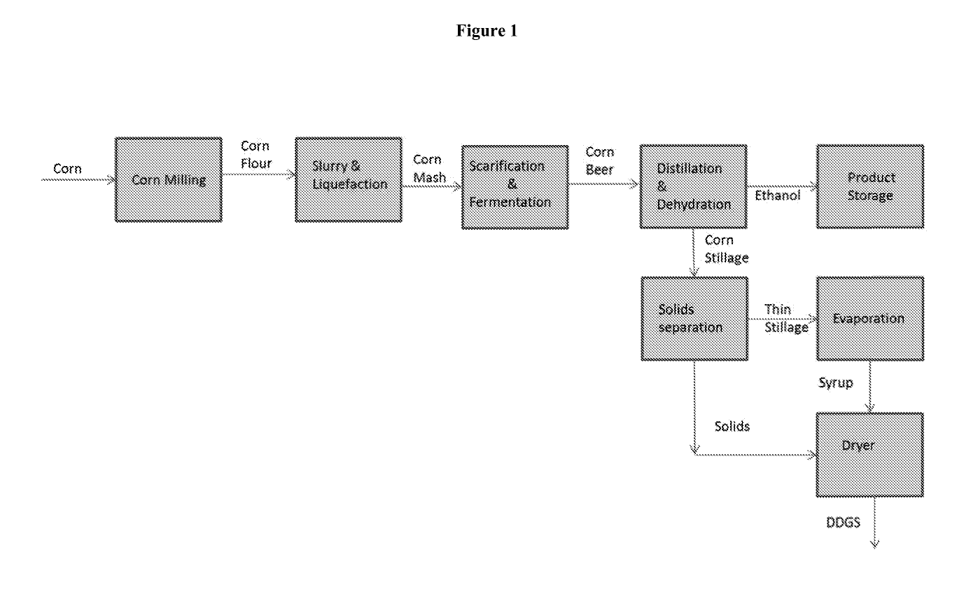

FIG. 2 is an embodiment of a process described herein using wet fractionation.

FIG. 3 is an embodiment of a process described herein using both dry and wet fractionation, with liquefaction performed after protein separation.

FIG. 4 is an embodiment of a process described herein using both dry and wet fractionation, with liquefaction performed before protein separation.

FIG. 5 is an embodiment of a process described herein using wet fractionation and a first and second pretreatment reactor.

FIG. 6 is graph showing ethanol concentration in a cofermentation process using native and engineered yeast strains.

FIG. 7 is a bar graph showing the concentration of ethanol and various sugars after 68 hours of fermentation using native and engineered yeast strains.

FIG. 8 is a graph depicting the mass and yield of CBH2 generated during a fermentation of xylose and glucose.

FIG. 9 is a graph depicting ethanol production in fermentations utilizing maltodextrin alone or maltodextrin and pretreated hardwood (PHW).

### DETAILED DESCRIPTION OF THE INVENTION

The articles “a,” “an,” and “the” are used herein to refer to one or to more than one (i.e., to at least one) of the grammatical object of the article. By way of example, “an element” means one element or more than one element.

The phrase “C5-enriched” means hemicellulose sugar enriched, particularly xylose and arabinose enriched. When the phrase C5-enriched is used to modify another term, e.g. “C5-enriched material,” it is to be understood that object described as C5-enriched can further include other components, such as, but not limited to, lignin and degradation products, such as acetic acid, furfural, and hydroxymethylfurfural (“HMF”).

The phrase “C5-enriched material” or “C5-enriched materials” refers to an aqueous slurry of solids resulting from the pretreatment of a cellulosic feed supply.

The phrase “hemicellulose sugar” means C5 sugars such as xylose and arabinose, and C6 sugars such as glucose, mannose, and galactose. In particular embodiments described herein, hemicellulose sugar can comprise at least about 70%, at least about 80%, at least about 90%, at least about 93%, at least about 95%, or at least about 97% xylose and arabinose, by weight, of the total sugar concentration, while the remaining sugars can be, for example, a combination of C6 sugars such as glucose, mannose, and/or galactose (generated by pretreatment of a feed supply).

As used herein the phrase “cellulosic feed supply” or “cellulosic feed” refers to any lignocellulosic material suitable for pretreatment. Examples of lignocellulosic materials suitable for pretreatment include, but are not limited to: cord grass, rye grass, reed canary grass, miscanthus, sugar-processing residues, sugarcane bagasse, agricultural wastes, rice straw, rice hulls, barley straw, corn cobs, cereal straw, wheat straw, canola straw, oat straw, oat hulls, soybean stover, forestry wastes, recycled wood pulp fiber, paper sludge, sawdust, hardwood, softwood, wood chips, corn fiber, corn stover, Panicum virgatum (switch grass), agave, the byproducts of lawn and tree maintenance, and combinations of any of the foregoing.

As used herein the term “maltodextrin” means the (C₆H₁₀O₅)ₙ.H₂O nonsweet, nutritive saccharide polymers that consist of D-glucose units linked primarily by alpha-1-4 bonds, having a dextrose equivalent (“DE”) of less than about 20. Maltodextrin has the CAS Reg. No. 9050-36-6.

As noted previously, in order to obtain the benefits of an integrated corn/cellulosic ethanol process, it is necessary to separate edible corn components from materials destined for combination with C5-enriched materials resulting from pretreatment of a cellulosic feed supply. As described in detail herein, isolating edible corn components can be accomplished in at least three ways: a) isolating edible corn components using wet fractionation and subsequently subjecting starch isolated from the wet fractionation process to liquefaction before combining it with C5-enriched materials for cofermentation; b) subjecting corn to a dry fractionation to separate germ and fiber from each other and from endosperm, subjecting the resulting endosperm to a wet fractionation to separate starch from protein in the endosperm, and subsequently subjecting the starch isolated from the wet fractionation process to liquefaction before combining it with C5-enriched materials for cofermentation; and c) subjecting corn to a dry fractionation to separate germ and fiber from each other and endosperm, subjecting the resulting endosperm to liquefaction to convert starch in the endosperm to maltodextrin, and subsequently separating protein (from the endosperm) from the maltodextrin before combining the maltodextrin with C5-enriched materials for cofermentation.

Each of these processes has the benefit of isolating edible corn components before these materials can be contaminated with impurities such as lignin, HMF, furfural, etc., thereby maintaining the value of the edible corn components such that these materials can be sold individually, as co-products, or on the DDGS market. Moreover, each provides options and opportunities for integration with existing corn-ethanol infrastructure, thereby reducing the cost of implementation.

### Fractionation

Fractionation, as either a wet or dry process, is a commercially well understood technology useful for separating corn into its component parts. Both wet and dry fractionation processes are able to separate corn fiber and germ from each other and from endosperm. Endosperm contains the starch useful for fermentation and protein useful for animal feed. Wet fractionation is further capable of separating protein in the endosperm from corn starch.

In certain embodiments, wet fractionation entails steeping corn in an aqueous solution optionally including SO₂ or an enzyme. Following the steeping process, germ can be separated from endosperm (starch and protein) and fiber. The aqueous solution from the steep, often referred to as “corn steep liquor,” can be sold or used during the fermentation process, optionally after removal of SO₂, if present. In certain embodiments, the starch, protein, and fiber can be combined in a slurry and can be passed to a milling station, which reduces the particle sizes of the various components of the mixture. Milling allows for ready separation of the fiber from the starch and protein. In certain embodiments, following fiber separation, the protein and starch are separated using a centrifuge such as a hydrocyclone—though other techniques are known in the art. This produces a protein stream and a starch stream, both of which can be further processed as desired using known techniques in the art.

For a further discussion of fractionation techniques, see, e.g., Murthy, G., et al, “Evaluation of a dry corn fractionation process for ethanol production with different hybrids,” Industrial Crops and Products, 2009 January 29(1):67-72; Singh, Vijay, “Corn Fractionation for Dry Grind Ethanol Production” Near-term Opportunities for Biorefineries Symposium, I-Hotel and Conference Center, Champaign, Ill. Oct. 11-12, 2010; Murthy, G., et al, “Evaluation and Strategies to Improve Fermentation Characteristics of Modified Dry-Grind Corn Processes,” Cereal Chemistry, 2006 September/October 83(5):455-459; Eckhoff, Steven R., and Stanley A. Watson. “Corn and Sorghum Starches: Production.” Starch: Chemistry and Technology. Ed. James N. BeMiller and Roy L. Whistler. 3rd ed. London: Academic, 2009. 374-439; and Johnson, Lawrence A., and James May. Corn: Chemistry and Technology. Ed. Pamela J. White and Lawrence A. Johnson. St. Paul, Minn.: American Association of Cereal Chemists, 2003. Each of the foregoing documents is incorporated herein by reference in its entirety.

### Liquefaction

Liquefaction is the process by which corn starch is converted to maltodextrin. During liquefaction, a corn flour slurry, comprising corn starch, water, and, optionally other materials such as protein from the endosperm, is cooked in the presence of a heat-stable enzyme, such as an α-amylase, at a temperature above about 100° C. in the presence of a shearing force. The heating and shearing of the cooking process breaks apart starch granules present in the corn flour, and the heat-stable enzyme breaks down the starch into maltodextrin. See, e.g., Wang, P., et al. “Comparison of Raw Starch Hydrolyzing Enzyme with Conventional Liquefaction and Saccharification Enzymes in Dry-Grind Corn Processing.” Cereal Chemistry 2007 January/February 84(1):10-14, the entirety of which is hereby incorporated by reference. Other materials, such as any protein present in the slurry, are largely unaffected by the liquefaction process.

### Pretreatment

In certain embodiments, a cellulosic feed supply is fed to a first pretreatment reactor wherein hemicellulose already present in the cellulosic feed supply is hydrolyzed. This pretreatment process generates a C5-enriched material that comprises an aqueous fraction and a solid fraction. The aqueous fraction comprises hemicellulose sugars, such as xylose and arabinose, as well as some C6 sugars present in lower amounts, such as glucose, mannose, and galactose. The aqueous fraction further includes acetic acid and degradation products such as furfural and hydroxymethylfurfural (“HMF”). The solid fraction includes cellulosic materials not completely hydrolyzed during the pretreatment process as well as lignin, acetate, and certain amounts of insoluble degradation products that are not well characterized.

Exemplary pretreatment conditions include any water based pretreatment approach including steam explosion pretreatment, hot water pretreatment, flow through pretreatment, and acid-based pretreatment (including acid catalyzed steam explosion, hot water and acid pretreatment, or flow through acid pretreatment). A more complete discussion of pretreatment conditions can be found in Kaar, et al, Steam Explosion Of Sugarcane Bagasse As A Pretreatment For Conversion To Ethanol, Biomass and Bioenergy Vol. 14, No. 3, pp. 277-287, 1998; and Mosier, Nathan, Charles Wyman, Bruce Dale, Richard Elander, Y. Y. Lee, Mark Holtzapple, and Michael Ladisch. Features of promising technologies for pretreatment of lignocellulosic biomass. Bioresource Technology 96 (2005) 673-686, each of which is incorporated herein by reference in its entirety.

The pretreatment reaction can be carried out in a number of reactor configurations including a horizontal Pandia-type configuration, or a vertical reactor configuration. Reactor configuration depends upon pretreatment type, feedstock type, desired performance, and energy consumption requirements.

In certain embodiments, the first pretreatment reactor can be run at a severity of about 3.7 to about 4.1, often noted as “low severity” conditions. This severity range corresponds to a temperature range of from about 180° C. to about 200° C. with a residence time of from about 5 minutes to about 30 minutes in a given reactor. Typically, low severity pretreatment is sufficient to solubilize hemicellulose in the cellulosic feed supply while minimizing degradation of the hemicellulose. In particular embodiments, the reactor can be a Pandia-type reactor.

In certain embodiments, the solid fraction can be separated from the liquid fraction (with additional extractive washes performed as necessary to remove soluble materials from the solid fraction) and fed to a second pretreatment reactor. The second pretreatment reactor can pretreat the solid fraction at a severity of about 4.1 to about 4.4 (corresponding to a temperature range of about 200° C. to about 220° C. and 5-15 minutes of residence time) resulting in a material suitable for combining with a fermentation feed for subsequent passage to a fermentation reactor.

### Cofermentation

In certain embodiments, the C5-enriched material (including both the aqueous and solid fraction or, optionally, only the aqueous fraction of the C5-enriched material) can be combined with the maltodextrin from any of the processes described herein, resulting in a fermentation feed. The fermentation feed can then be passed to a fermentation reactor, at a specified rate, for cofermentation.

In certain embodiments, the fermentation feed can have a concentration of hemicellulose sugars and maltodextrin (“total sugar concentration”) of about 100 g/L to about 500 g/L. In other embodiments, the fermentation feed can contain a concentration of about 150 g/L to about 450 g/L total sugar concentration. In still other embodiments, the fermentation feed can have a concentration of about 200 g/L to about 400 g/L total sugar concentration. In another embodiment, the fermentation feed can have about 250 g/L to about 350 g/L total sugar concentration. And in a further embodiment, the fermentation feed can have about 300 g/L total sugar concentration.

In the fermentation feed, the maltodextrin can comprise from about 50% to about 95% by weight of the total sugar concentration. In other embodiments, the maltodextrin can comprise from about 70% to about 90% by weight of the total sugar concentration. In other embodiments, the maltodextrin can comprise from about 80% to about 85% by weight of the total sugar concentration. In a particular embodiment, the maltodextrin can comprise about 83% by weight of the total sugar concentration.

In a particular embodiment, the fermentation feed can have about 300 g/L total sugar concentration, of which about 83% (i.e. about 250 g/L) can be maltodextrin.

Although the fermentation feed can have a concentration in any of the ranges noted above, sugar concentration in the fermentation reactor will ultimately be less than the concentration of the fermentation feed and will vary based on at least the rate at which the fermentation feed is fed into the reactor, the rate at which yeast in the reactor converts fermentable sugars to ethanol, the rate at which cellulose present in the solid fraction of the C5-enriched material (if present) is broken down into soluble simple sugars, the concentration of inhibitors in the fermentation feed and/or reactor, and other factors known to those of ordinary skill in the art.

The quantity of C5-enriched material combined with the maltodextrin allows an operator to control the quantity of inhibitors in the fermentation feed and fermentation reactor. For example, and as discussed earlier, the C5-enriched material, in addition to containing hemicellulose sugars, also contains undesirable byproducts from the pretreatment process. These undesirable byproducts include, but are not limited to, lignin, furfural, HMF (hydroxymethylfurfural), and acetic acid.

Acetic acid, for example, acidifies yeast cytosol and requires the yeast to use ATP to pump protons out of the cell. This stresses the micro-organism and inhibits the production of ethanol. See, for example, Bellissimi E., et al., “Effects of acetic acid on the kinetics of xylose fermentation by an engineered, xylose-isomerase-based Saccharomyces cerevisiae strain,” FEMS Yeast Res. 2009 May; 9(3):358-64, the entirety of which is hereby incorporated by reference. The inhibitor effects of lignin are likewise well known. See, for example, Klinke H. B., et al., “Inhibition of ethanol-producing yeast and bacteria by degradation products produced during pre-treatment of biomass,” Appl. Microbiol. Biotechnol. 2004 November; 66(1):10-26, the entirety of which is hereby incorporated by reference.

Traditional approaches to minimizing these undesirable impurities in pretreated materials included, but were not limited to, optimizing pretreatment conditions, removing some acetic acid and/or furfural through evaporation prior to fermentation, addition of makeup water, and engineering robust organisms capable of handling high concentrations of inhibitors. Despite these measures, it was still often necessary to run fermentations at concentrations of about 120 g/L to about 140 g/L, and in certain embodiments, at about 130 g/L for hemicellulose sugars streams derived from a cellulosic feed supply subject to pretreatment.

But, by controlling the ratio of the C5-enriched material and maltodextrin in the fermentation feed, the concentration of impurities in the fermentation feed, and thus the fermentation reactor, can also be controlled. For example, in certain embodiments, the combined concentration of HMF and furfural in the fermentation feed can be less than about 1 g/L, less than about 0.5 g/L, less than about 0.2 g/L, less than about 0.1 g/L, less than about 0.01 g/L, less than about 0.001 g/L, or less than about 0.0001 g/L all while maintaining the total initial sugar concentration discussed elsewhere herein. In particular embodiments, the combined concentration of HMF and furfural can be about 1 g/L.

Similarly, in certain embodiments, the concentration of acetic acid in the fermentation feed can be less than about 20 g/L, less than about 15 g/L, less than about 10 g/L, less than about 5 g/L, or less than about 1 g/L. In particular embodiments, the acetic acid concentration in the fermentation feed can be about 4 g/L.

In certain embodiments lignin concentration in the fermentation feed can be less than about 70 g/L, less than about 50 g/L, less than about 30 g/L, less than about 25 g/L, less than about 20 g/L, less than about 18 g/L, less than about 16 g/L, or less than about 10 g/L. In particular embodiments, the lignin concentration in the fermentation feed can be about 16 g/L.

Thus, by combining the C5-enriched materials and the maltodextrin in the appropriate ratios, a skilled artisan can control impurity concentration in the fermentation feed and increase the overall efficiency of a given fermentation, resulting in increased ethanol production.

In certain embodiments, a glucoamylase can be added to the fermentation feed prior fermentation to reduce the viscosity of the fermentation feed and/or to reduce maltodextrin inhibition of cellulases and hemicellulases present in the fermentation reactor. In other embodiments, the yeast used to ferment the fermentation feed can produce glucoamylase in situ.

The fermentation feed can be fermented in the fermentation reactor in the presence of one or more yeast strains disclosed in U.S. Pat. No. 8,470,592; U.S. Pat. No. 8,658,398; U.S. Ser. No. 14/188,360; U.S. Ser. No. 13/130,549; U.S. Ser. No. 13/141,952; U.S. Ser. No. 13/514,519; U.S. Ser. No. 13/459,804; U.S. Ser. No. 13/640,223; U.S. Ser. No. 13/391,554; U.S. Ser. No. 13/696,207; U.S. Ser. No. 13/814,616; U.S. Ser. No. 14/110,075; U.S. Ser. No. 14/075,846; U.S. 61/866,338; U.S. Ser. No. 12/677,428; U.S. Ser. No. 12/992,001; U.S. Ser. No. 12/599,425; U.S. Ser. No. 12/992,003; U.S. Ser. No. 13/124,255; U.S. Ser. No. 13/201,257; U.S. Ser. No. 13/393,093; PCT/US2011/039192; PCT/US2011/066968; PCT/US2012/064457; PCT/US2012/057952; PCT/US2012/067216; PCT/US2013/070964; PCT/US2013/000090, PCT/US2014/025460; and/or US 2013/0323822, the entireties of which are incorporated herein by reference.

Cellulolytic enzymes can be added to the fermentation reactor to supplement the activity of any of the yeasts described herein. Examples of these cellulolytic enzymes are disclosed in, for example, Nigam, P. S., et al. Biotechnology for Agro-industrial Residues Utilisation: Utilisation of Agro-residues. Netherlands: Springer, 2009; Zhang, Yi-Heng Percival, et al. Towards an Aggregated Understanding of Enzymatic Hydrolyis of Cellulose: Noncomplexed Cellulase systems. Wiley Interscience, 2004; and Bhat, M. K. Cellulases and related enzymes in biotechnology. Biotechnology Advances 18 (2000) 355-383—each of which is incorporated herein by reference in its entirety.

Fermentation (cofermentation) can be carried using conditions similar to and consistent with traditional corn ethanol fermentation conditions. Thus, in certain embodiments, the reactor is a fed-batch reactor. In certain embodiments, the fermentation can be carried out over a period of about 50 to about 100 hours, about 55 to about 90 hours, or about 60 to about 80 hours. In certain embodiments, total weight of solids in the fermentation reactor can range from about 1 to about 50%, from about 10% to about 40%, or from about 20% to about 30% by weight. Fermentation as discussed herein results in a beer having an ethanol titration after fermentation ranging from about 5% to about 30% by weight, about 5% to about 20% by weight, about 5% to about 15% by weight, or, in certain embodiments, from about 8% to about 14% by weight.

The beer can then be distilled, using known techniques, resulting in an ethanol stream. In certain embodiments, the ethanol stream can then be dehydrated using known techniques, such as azeotropic distillation or molecular sieves. See, e.g., Bastidas, P., et al. “Comparison of the main ethanol dehydration technologies through process simulation” 20th European Symposium on Computer Aided Process Engineering, 2010, the entirety of which is hereby incorporated by reference.

### Integrated Embodiments

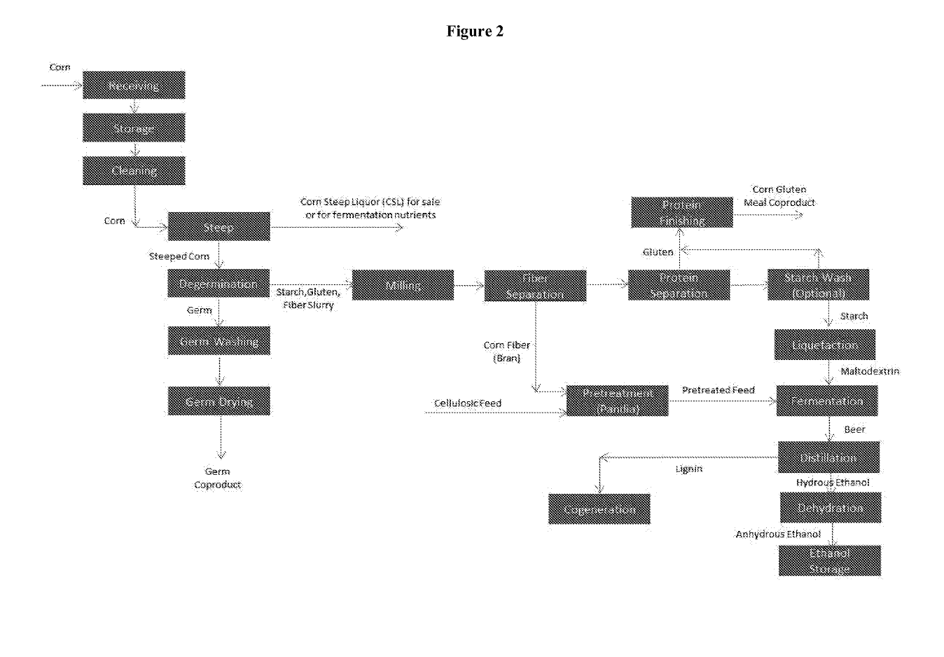

In one embodiment, an example of which is shown in FIG. 2, a standard wet fractionation technique can be employed. In this embodiment, corn (after undergoing appropriate cleaning) can be steeped in an aqueous solution optionally including SO₂ or an enzyme. Following the steeping process, germ can be separated from fiber and endosperm (starch and protein/gluten). The germ can then be washed and dried for storage and sale.

The aqueous solution from the steep, or “corn steep liquor,” can be sold or used during the fermentation process, both after removal of SO₂, if present. The starch, protein (gluten), and fiber can then be passed to a milling station as a slurry in water. The milling reduces the particle sizes of the various components of the mixture and allows for mechanical separation of the fiber from the starch and protein/gluten. The separated fiber can be stored (not shown in FIG. 2) or processed in a pretreatment reactor.

Once separated from the fiber, the protein/gluten and starch can be separated using known techniques. In certain embodiments, a centrifuge such as a hydrocyclone can be employed. This produces a protein/gluten stream and a starch stream. The protein/gluten stream can be passed to finishing (concentrating and drying), while the starch stream can be passed to an optional washing stage before being passed to liquefaction where the starch is converted to maltodextrin.

Post liquefaction, the resulting maltodextrin can be combined with C5-enriched materials resulting from the pretreatment of a cellulosic feed. In certain embodiments, the pretreatment can occur in a Pandia® type reactor. The cellulosic feed can optionally include fiber (bran) from the earlier fiber separation stage. The combined maltodextrin and C5-enriched materials are called a fermentation feed.

Although not shown in FIG. 2, a glucoamylase can optionally be added to the fermentation feed, using, for example, a commercial product or a recombinant microorganism, to reduce the viscosity of the fermentation feed and/or maltodextrin inhibition of the yeast in the cofermentation. The fermentation feed can then be fermented and subsequently processed to produce anhydrous ethanol.

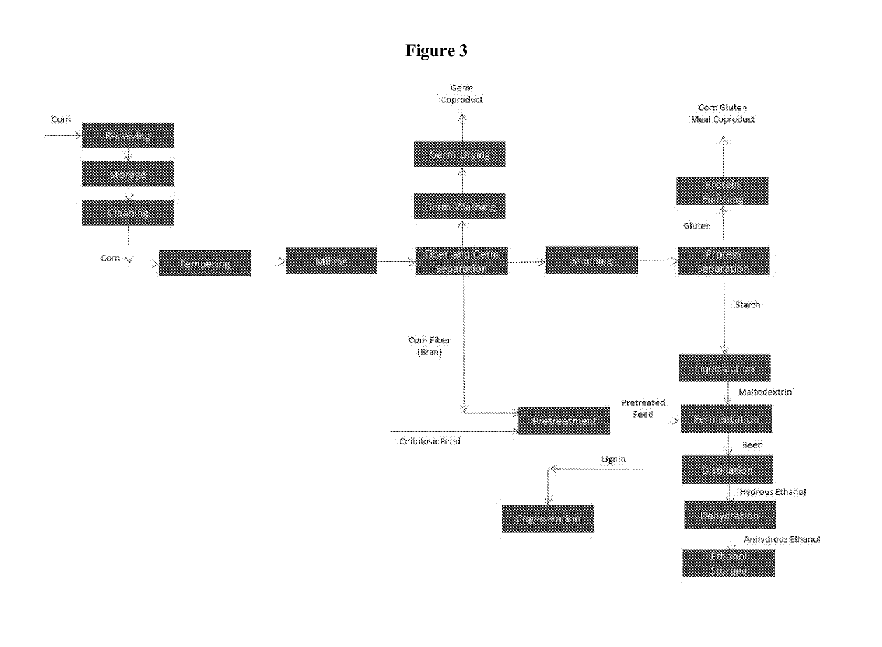

In another embodiment, an example of which is shown in FIG. 3, corn (after undergoing appropriate cleaning) can be tempered. In particular embodiments, the corn can be tempered using known techniques to about 10%, about 15%, about 18%, or about 20% moisture content. The tempered corn can then be passed to a milling operation to reduce the size of the tempered corn. Following milling, the resulting material can be passed to fiber and germ separation stage which separates fiber, germ, and endosperm (starch and protein/gluten) from each other. The germ can then be washed and dried for storage and sale. Fiber (bran) can be processed, stored for later use, or sold as discussed previously.

The endosperm can then be steeped in an aqueous solution optionally including SO₂ or an enzyme. Following steeping starch and protein/gluten in the endosperm can be separated from each other. The protein/gluten can be passed to finishing (concentrating and drying), while the starch can be passed to liquefaction. The aqueous solution from the steep, or “corn steep liquor,” can be sold or used during the fermentation process, both after removal of SO₂, if present.

Post liquefaction, the resulting maltodextrin can be combined with C5-enriched materials resulting from the pretreatment of a cellulosic feed. In certain embodiments, the pretreatment can occur in a Pandia® type reactor. The cellulosic feed can optionally include fiber (bran) from the earlier fiber separation stage. As before, the combined maltodextrin and C5-enriched materials are called a fermentation feed.

Although not shown in FIG. 3, a glucoamylase can optionally be added to the fermentation feed. The fermentation feed can then be passed to a fermentation reactor where it is fermented. The resulting ethanol can then be distilled and dried.

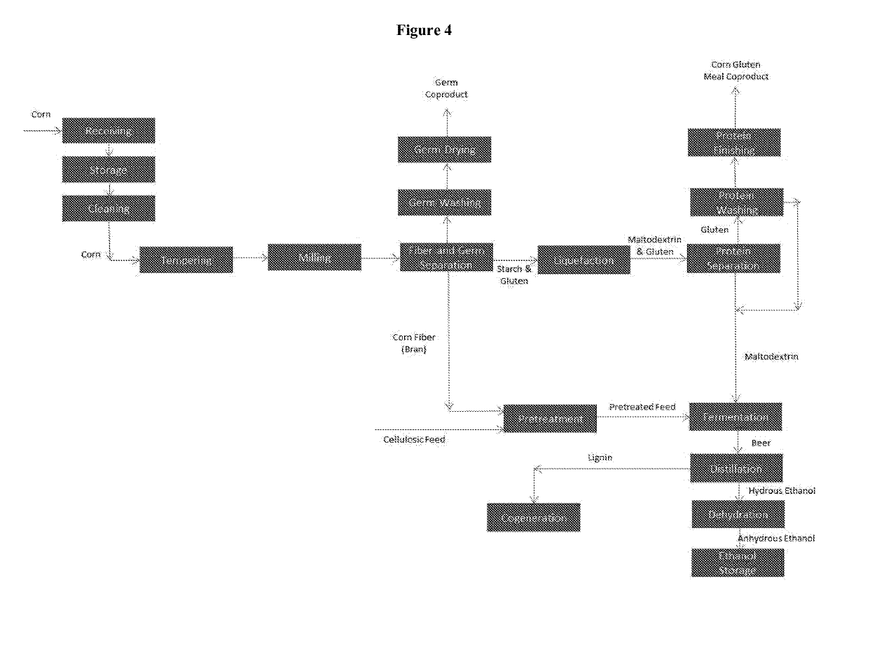

In a further embodiment, an example of which is shown in FIG. 4, corn (after undergoing appropriate cleaning) can be tempered. In particular embodiments, the corn can be tempered using known techniques to about 10%, about 15%, about 18%, or about 20% moisture content. The tempered corn can then be passed to a milling operation to reduce the size of the tempered corn. Following milling, the resulting material can be passed to fiber and germ separation stage which separates fiber, germ, and endosperm (starch and protein/gluten) from each other. The germ can then be washed and dried for storage and sale. Fiber (bran) can be processed, stored for later use, or sold as discussed previously.

The endosperm can then be passed to liquefaction to produce a maltodextrin and protein/gluten stream. The maltodextrin and protein/gluten stream can then be passed to a protein separation stage, typically a hydrocyclone or other centrifugation process capable of separating the insoluble protein from the soluble maltodextrin. This process results in a maltodextrin stream and a protein stream. Once separated, the protein stream can optionally be washed as appropriate to remove any residual maltodextrin. The washings can optionally being combined with the maltodextrin stream. The protein can then be passed to finishing (concentrating and drying).

Post separation, the maltodextrin stream can be combined with C5-enriched materials resulting from the pretreatment of a cellulosic feed. In certain embodiments, the pretreatment reactor is a Pandia® type reactor. The cellulosic feed can optionally include fiber (bran) from the earlier fiber separation stage. The combined maltodextrin and C5-enriched materials are called a fermentation feed.

Although not shown in FIG. 4, a glucoamylase can optionally be added to the fermentation feed. The fermentation feed can then be passed to a fermentation reactor for the production of ethanol. Ethanol resulting from fermentation can then be processed to produce anhydrous ethanol using the procedures disclosed herein.

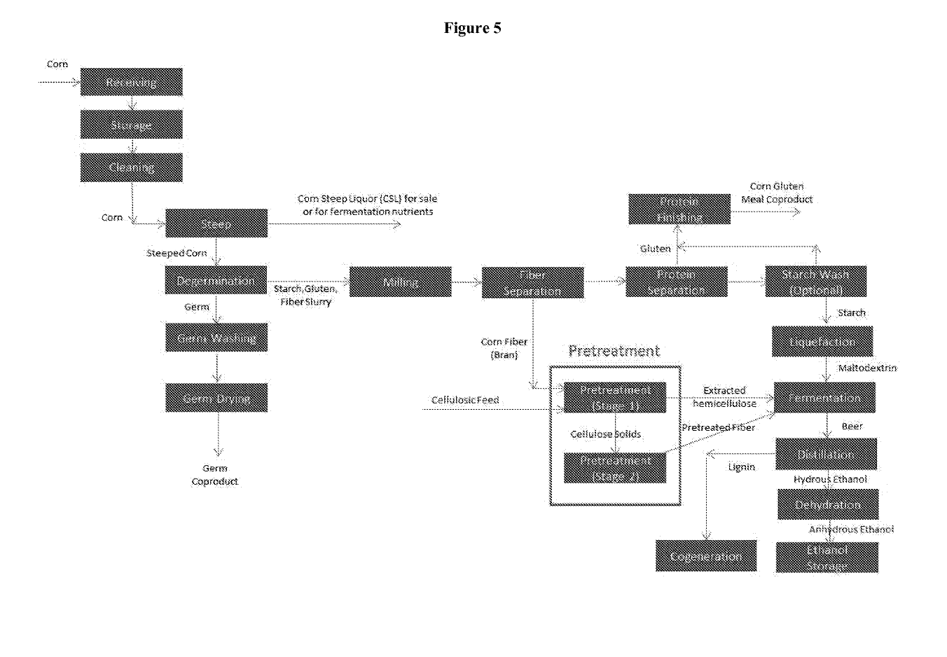

In another embodiment, an example of which is shown in FIG. 5, and which is a variant of the embodiment shown in FIG. 2, a standard wet fractionation technique can be employed. But, in this embodiment, the maltodextrin resulting from liquefaction can be combined with the aqueous fraction of the C5-enriched materials resulting from a first pretreatment of a cellulosic feed to give a fermentation feed, while the solid fraction of the C5-enriched materials can be passed to a second pretreatment reactor for further pretreatment. The materials resulting from this further pretreatment can then be combined with the fermentation feed and the resulting mixture can then be fed to a fermentation reactor for further processing to ethanol.

### Integrated Corn Ethanol Processing Plants

In addition to the methods described herein, the present disclosure further provides integrated corn ethanol processing plants. In one embodiment, an ethanol processing plant includes: a steeping stage; a degermination stage; a milling stage; a fiber separation stage; a protein separation stage; a liquefaction stage; and a fermentation stage; wherein the degermination stage separates starch, protein, and fiber from germ; the fiber separation stage separates fiber from the starch and protein; the protein separation stage separates the protein from the starch; and the fermentation stage is adapted to receive maltodextrin produced in the liquefaction stage and C5-enriched materials produced during pretreatment of a cellulosic feed supply. The plant can further include one or more pretreatment reactors.

In another embodiment, an ethanol processing plant comprises a tempering stage; a milling stage; a fiber and germ separation stage; a steeping stage; a protein separation stage; a liquefaction stage; and a fermentation stage. In particular embodiments, the tempering stage hydrates corn to a selected water content; the fiber and germ separation stage separates fiber and germ from each other and from endosperm; the steeping stage steeps the endosperm isolated during the fiber and germ separation stage to produce a steeped endosperm; the protein separation stage separates protein from starch in the steeped endosperm; and the fermentation stage is adapted to receive maltodextrin produced in the liquefaction stage and C5-enriched materials produced during pretreatment of a cellulosic feed supply. The plant can further include one or more pretreatment reactors.

In still another embodiment of an integrated processing plant, the processing plant comprises a tempering stage; a milling stage; a fiber and germ separation stage; a liquefaction stage; a protein separation stage; and a fermentation stage. In particular embodiments, the tempering stage dries corn to a selected water content; the fiber and germ separation stage separates fiber and germ from each other and from endosperm; the liquefaction stage reduces starch in the endosperm to maltodextrin; the protein separation stage separates protein from maltodextrin produced during the liquefaction stage; and the fermentation stage is adapted to receive the maltodextrin and C5-enriched materials produced during pretreatment of a cellulosic feed supply. The plant can further include one or more pretreatment reactors.

### EXAMPLES

The processes described herein are now further detailed with reference to the following examples. These examples are provided for the purpose of illustration only and the processes described herein should in no way be construed as being limited to these examples. Rather, the processes should be construed to encompass any and all variations which become evident as a result of the teaching provided herein.

### Example 1

### Cofermentation

Native yeast (strain M2390) which is not capable of consuming xylose, was compared to xylose utilizing strain M4638 (see, e.g., PCT/US2013/000090) in a cofermentation reaction. In each cofermentation, 20 g/l xylose was mixed with 28% total solids corn mash sourced from a commercial operation (standard corn mash containing all corn components including nonfermentables) and fermented for 72 hours in a 50 mL batch shake flask fermentation. Spirizyme Excel (Novozymes)—a glucoamylase—was dosed at a loading of 0.6 AGU (amyloglucosidase units) per gram of total solids, and urea was added as a nitrogen source at a loading of 1000 ppm. Yeast strains M2390 and M4638 were each precultured on YPD (10 g/L yeast extract, 20 g/L peptone, and 20 g/L dextrose) media for 24 hours, and inoculated at 0.3 g DCW/L. The fermentation temperature was held at 35° C. for the first 24 hours, and then shifted to 32° C. thereafter.

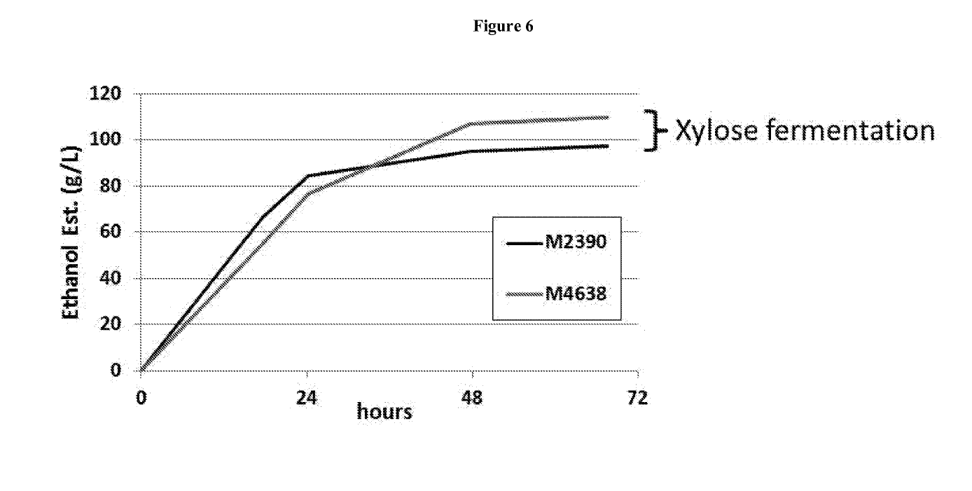

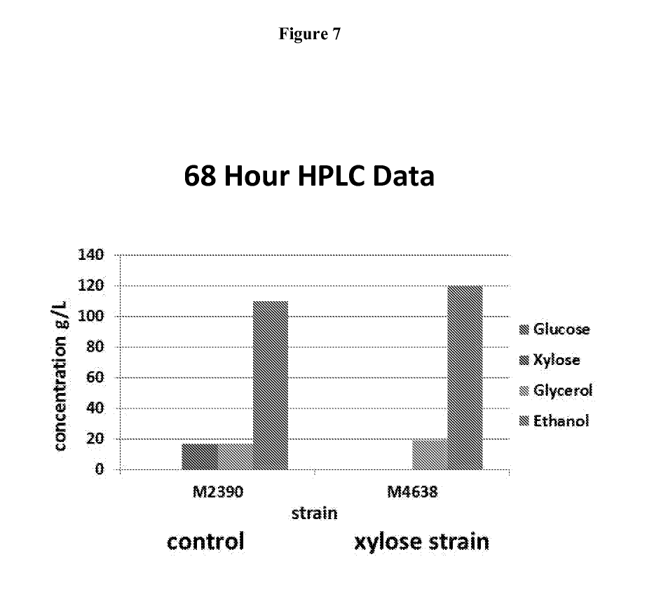

As is shown in FIGS. 6 and 7, M4638 showed that it was possible to coferment a mixture of xylose and glucose in the presence of certain insoluble materials. In particular, it was observed that the xylose was consumed along with glucose within a typical corn fermentation time frame of 68 hrs., indicating that reaction kinetics were sufficient to permit complete fermentation in the allotted time. A higher ethanol titer was observed for the strain that could consume xylose, indicating a yield improvement using the cofermentation.

### Example 2

### CBH2 Enzyme Production

An experiment to measure the impact of glucose feeding on enzyme expression for certain yeast strains was carried out with CBH2 expressing strain M1873 (See, e.g., PCT/US2011/039192). This strain was cultured in a 2 L working volume Sartorius A+ bioreactor with temperature and pH control. Media used in the experiment was a combination of corn steep liquor (CSL) loaded at 12 g/L (wet weight, 50% solids), diammonium phosphate (DAP) loaded at 2.2 g/L on a dry basis, and magnesium sulfate heptahydrate loaded at 2.46 g/L. Media was also supplemented with pressate from pretreated hardwood (PHW) material to simulate the presence of inhibitors. The PHW material, MS801W, was generated using a pilot scale continuous pretreater by applying steam to red maple hardwood chips to achieve a temperature of 195° C. for 10 minutes. The chips had been soaked in maleic acid resulting in 0.5% w/w % maleic acid in the pretreater. The material was steam exploded and washed with water at a 20:1 liquid to solid ratio to generate washed solids. This material was then pressed using a lab press to create a liquor with inhibitors used to simulate the concentration of soluble inhibitors in a 22% total solids PHW fermentation. 330.6 g of this material was added to the reactor with the glucose feed and contained 1.2 g/L xylose as the only soluble sugar. The reaction was carried out as a fed-batch culture, with an initial volume of 500 mL containing 2.5 g of glucose, and media components as described above, loaded on a final fermentation (1 L) volume basis. A feed of glucose mixed with MS801W pressate was fed to this initial volume and contained 91.4 g of glucose, in a total volume of 489 mL (330.6 mL of PHW pressate and 158.4 mL of water). The rate of feeding was scheduled as follows: 0.12 mL/min for the first 24 hours, 0.06 mL/min from 24 to 90 hours, and 0.05 mL/min until the end of the reaction at 122 hours.

The amount of ethanol and sugars were tracked over time via HPLC analysis using the Biorad Aminex 87P column and appropriate standards. Concentration of CBH2 in media was performed by a phenyl reversed phase HPLC method using an Agilent 2100 HPLC with the MWD detector at 214 and 280 nm. In this method, purified CBHs were used for generating a standard curve from 200-10 μg. The sample was injected onto a phenyl RP column (Tosoh phenyl-5PW RP, 4.6 mm×7.5 cm, 10 μm) that was equilibrated at 55° C. in 0.1% trifluoracetic acid (TFA) (w/v), 20% acetonitrile. The CBH2 was eluted from the column at 0.75 ml/min using a linear gradient of acetonitrile with 0.1% TFA (w/v) from 20-60% in 45 minutes. After cleaning the column with 95% acetonitrile/TFA, the column was re-equilibrated. To determine the concentration of CBH2 produced in media by various strains, the peak area of the sample was compared to the standard curve generated from the peak areas of the purified CBH2 (μg/μL injected).

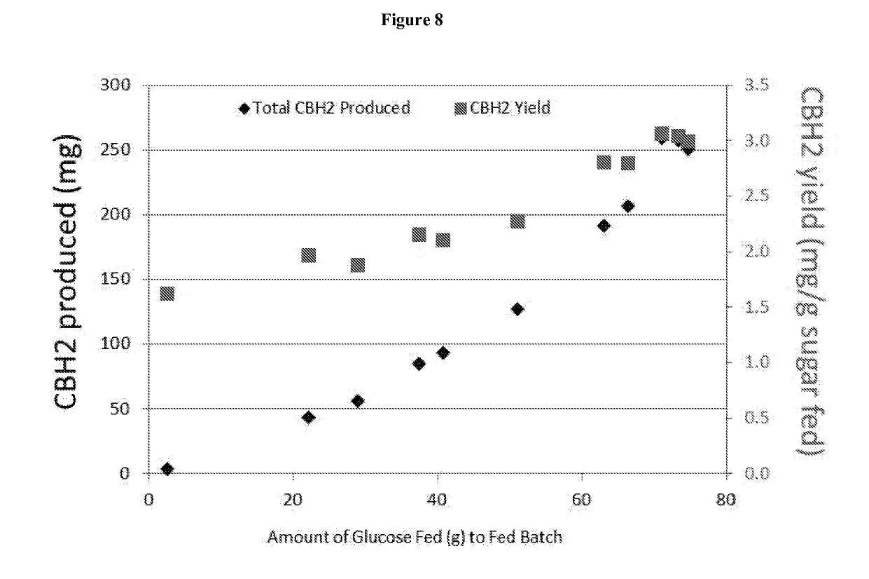

Results of this experiment are shown in FIG. 8. The total amount (mass in mg) of CBH2 produced as sugar was fed to the reactor is shown by the diamonds. By the end of the fermentation about 250 mg of CBH2 was made. The cumulative yield of CBH2 per gram of sugar fed to the reactor up to the sugar feed amount given on the x-axis is shown by the squares. Yield increases across the reaction from 1.6 mg/g sugar to 3 mg/g sugar. These results demonstrate that CBP strains fed with sugar continue to produce additional enzyme during fermentation, and that the yield of this enzyme produced per amount of sugar fed may in fact increase. Thus, cellulosic CBP fermentations fed with additional easily accessible sugars in the form of starch, maltodextrin, or glucose from corn, will benefit due to the additional cellulase (or other) enzymes produced.

### Example 3

### Cofermentation of Maltodextrin and Pretreated Hardwood

To directly test the idea that co-fermentation of maltodextrin and PHW would be beneficial, fermentations were carried out in sealed bottles with a combination of about 150 g/L maltodextrin both with and without 50 g/L PHW. Two strains were tested in this configuration: M2390, a wild type S. cerevisiae strain, and M3799, a xylose utilizing strain described in PCT/US2013/000090.

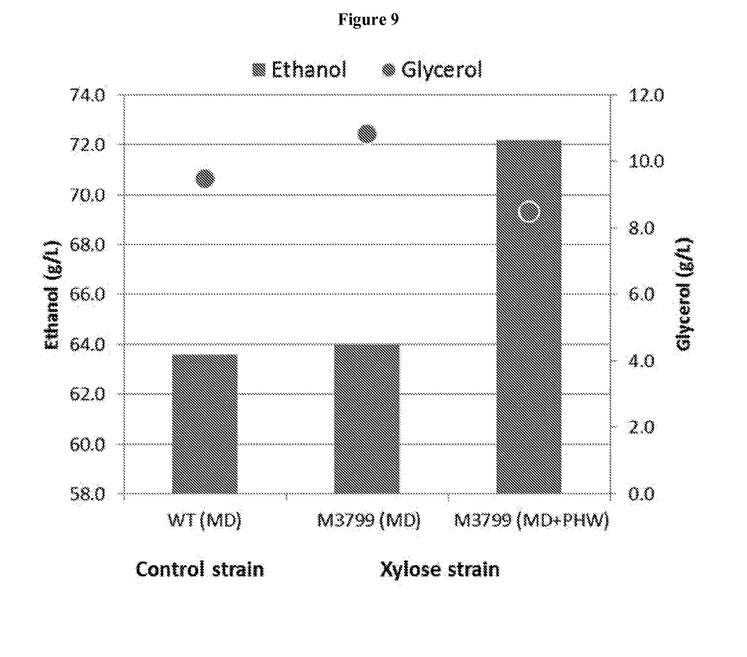

In control experiments, strains M2390 and M3799 were fed maltodextrin only at about 150 g/L. Maltodextrin was obtained from Tate & Lyle Ingredients. Spirizyme Excel was added to these fermentations at 0.6 AGU/g maltodextrin, and both strains produced about 64 g/L of ethanol in 48 hours. Approximately 10 g/L of glycerol was produced. See FIG. 9, where “WT” refers to “wild type” and “MD” refers to maltodextrin.

Next, a fermentation using M3799 was performed in which maltodextrin and PHW were included. PHW was created by pretreating aspen hardwood pulp chips using a continuous steam pretreater and applying steam to achieve a cook temperature of 182° C. for 25 minutes. In this pretreatment only steam was applied, no acid was used. The material was steam exploded out of the reactor to generate a fiber material, which was used directly (no washing). The material was characterized by quantitative saccharification as described in the standard published NREL procedure (NREL/TP-510-42618), and found to have 48.5% glucose (in the form of monomer and polymer glucan) and 18.5% xylose galactose and mannose (in the form of monomer and polymer hemicellulose).

To carry out the fermentation with PHW, the PHW materials described above were mixed with water to appropriate concentrations (considering dilution with media and inoculum). Next, media was added at final concentrations of: 12 g/L CSL (wet basis, 50% solids mixture), 1 g/L DAP, 10 g/L calcium carbonate (CaCO₃) for pH control, and penicillin at 50 μg/mL. Two types of enzymes were loaded: Spirizyme Excel as a glucoamylase at 0.6 AGU/g maltodextrin, and Flashzyme (AB Enzymes) as a cellulase at 4 mg enzyme per gram of total solids. Strains were inoculated at 0.5 g DCW/L.

FIG. 9 shows the effect of loading PHW at 50 g dry solids/L. More than 72 g/L of ethanol was produced in this case from the additional release of xylose and glucose from the PHW. About 8.5 g/L of glycerol was formed in this case. This data demonstrates conversion of about 47% of the loaded PHW into ethanol in 48 hours of fermentation time at a low enzyme dose of 4 mg/g total solids, representing about a 13% yield increase on the corn ethanol fermentation represented by the maltodextrin only fermentations.

The phraseology or terminology herein is for the purpose of description and not of limitation. As such, the terminology and/or phraseology of the present specification should be interpreted by the skilled artisan in light of the teachings and guidance herein.

The breadth and scope of the present invention should not be limited by any of the above-described exemplary embodiments, but should be defined only in accordance with the following claims and their equivalents.

What is claimed is:

1. A corn-ethanol production method comprising:
a. fractionating corn to produce corn starch;
b. converting the corn starch into maltodextrin; and
c. cofermenting hemicellulose sugars and the maltodextrin in a fermentation reactor in the presence of a yeast capable of fermenting hemicellulose sugar and glucose to ethanol;
wherein
the yeast is further capable of converting the maltodextrin to glucose or wherein the maltodextrin is treated with a glucoamylase prior to cofermentation.

2. The method of claim 1, wherein the fractionating is wet fractionating.

3. The method of claim 2, wherein the wet fractionating comprises steeping, degermination, milling, fiber separation, and protein separation.

4. The method of claim 3, wherein the steeping comprises soaking the corn in an aqueous solution optionally including SO₂ or an enzyme.

5. The method of claim 1, wherein converting the corn starch into maltodextrin comprises liquefaction.

6. The method of claim 5, wherein the liquefaction comprises cooking the corn starch in the presence of a heat-stable enzyme at a temperature above about 100° C. in the presence of a shearing force.

7. The method of claim 6, wherein the heat-stable enzyme is an α-amylase.

8. The method of claim 1, wherein the hemicellulose sugars are derived from at least a first pretreatment of a cellulosic feed optionally comprising corn fiber.

9. The method of claim 8, wherein the at least a first pretreatment has a severity of from about 3.7 to about 4.1.

10. The method of claim 1, wherein maltodextrin is treated with a glucoamylase prior to fermentation.

11. The method of claim 1, wherein before being fed to the fermentation reactor, the hemicellulose sugars and the maltodextrin comprise a fermentation feed having a total sugar concentration of about 100 g/L to about 500 g/L.

12. The method of claim 11, wherein the total sugar concentration is about 250 g/L to about 350 g/L.

13. The method of claim 12, wherein the total sugar concentration is about 300 g/L.

14. The method of claim 11, wherein maltodextrin comprises from about 50% to about 90% by weight of the total sugar concentration in the fermentation feed.

15. The method of claim 14, wherein the maltodextrin comprises about 83% by weight of the total sugar concentration in the fermentation feed.

16. The method of claim 1, wherein the fractionating comprises dry fractionating the corn to separate fiber and germ from endosperm.

17. The method of claim 16, wherein the dry fractionating comprises milling.

18. The method of claim 16, wherein the fractionating further comprises wet fractionating the endosperm to separate corn starch and protein present in the endosperm from each other.

19. The method of claim 18, wherein the wet fractionating comprises steeping.

20. The method of claim 19, wherein the steeping comprises soaking the corn in an aqueous solution optionally including SO₂ or an enzyme.

21. The method of claim 18, wherein converting the corn starch into maltodextrin comprises liquefaction.

22. The method of claim 21, wherein the liquefaction comprises cooking the corn starch in the presence of a heat-stable enzyme at a temperature above about 100° C. in the presence of a shearing force.

23. The method of claim 22, wherein the heat-stable enzyme is an α-amylase.

24. The method of claim 18, wherein the hemicellulose sugars are derived from the pretreatment of a cellulosic feed optionally comprising corn fiber.

25. The method of claim 24, wherein the pretreatment has a severity of from about 3.7 to about 4.1.

26. The method of claim 18, wherein the maltodextrin is treated with a glucoamylase prior to cofermentation.

27. The method of claim 18, wherein before being fed to fermentation reactor, the hemicellulose sugars and the maltodextrin comprise a fermentation feed having a total sugar concentration of about 100 g/L to about 500 g/L.

28. The method of claim 27, wherein the total sugar concentration is about 250 g/L to about 350 g/L.

29. The method of claim 28, wherein the total sugar concentration is about 300 g/L.

30. The method of claim 27, wherein maltodextrin comprises from about 50% to about 90% by weight of the total sugar concentration in the fermentation feed.

31. The method of claim 30, wherein the maltodextrin comprises about 83% by weight of the total sugar concentration in the fermentation feed.

32. A corn-ethanol production method comprising:
a. fractionating corn to produce a mixture of protein and corn starch;
b. converting the corn starch in the mixture of protein and corn starch to maltodextrin in the presence of the protein;
c. separating the maltodextrin from the protein; and
d. cofermenting hemicellulose sugars and the maltodextrin in a fermentation reactor in the presence of a yeast capable of fermenting hemicellulose sugar and glucose to ethanol
wherein
the yeast is further capable of converting the maltodextrin to glucose or wherein the maltodextrin is treated with a glucoamylase prior to cofermentation.

33. The method of claim 32, wherein converting the corn starch in the mixture of protein and corn starch to maltodextrin in the presence of the protein comprises liquefaction of the mixture of protein and corn starch.

34. The method of claim 33, wherein the liquefaction comprises cooking the corn starch and protein in the presence of a heat-stable enzyme at a temperature above about 100° C. in the presence of a shearing force.

35. The method of claim 34, wherein the heat-stable enzyme is an α-amylase.

36. The method of claim 32, wherein the hemicellulose sugars are derived from the pretreatment of a cellulosic feed optionally comprising corn fiber.

37. The method of claim 36, wherein the pretreatment has a severity of from about 3.7 to about 4.1.

38. The method of claim 32, wherein the maltodextrin is treated with a glucoamylase prior to cofermentation.

39. The method of claim 32, wherein before being fed to the fermentation reactor, the hemicellulose sugars and the maltodextrin comprise a fermentation feed having a total sugar concentration of about 100 g/L to about 500 g/L.

40. The method of claim 39, wherein the total sugar concentration is about 250 g/L to about 350 g/L.

41. The method of claim 40, wherein the total sugar concentration is about 300 g/L.

42. The method of claim 39, wherein maltodextrin comprises from about 50% to about 90% by weight of the total sugar concentration in the fermentation feed.

43. The method of claim 42, wherein the maltodextrin comprises about 83% by weight of the total sugar concentration in the fermentation feed.

44. The method of claim 32, wherein separating the maltodextrin and protein comprises centrifuging the protein away from the maltodextrin.

45. The method of claim 44, wherein the centrifuging comprises using a hydrocyclone.

46. An ethanol processing plant comprising:
a. a steeping stage;
b. a degermination stage;
c. a milling stage;
d. a fiber separation stage;
e. a protein separation stage;
f. a liquefaction stage; and
g. a fermentation stage;
wherein
the degermination stage separates starch, protein, and fiber from germ;
the fiber separation stage separates fiber from the starch and protein;
the protein separation stage separates the protein from the starch; and
the fermentation stage is adapted to receive a fermentation feed comprising maltodextrin produced in the liquefaction stage and C5-enriched materials produced during pretreatment of a cellulosic feed supply.

47. An ethanol processing plant comprising:
a. a tempering stage;
b. a milling stage;
c. a fiber and germ separation stage;
d. a steeping stage
e. a protein separation stage;
f. a liquefaction stage; and
g. a fermentation stage;
wherein
the tempering stage dries corn to a selected water content;
the fiber and germ separation stage separates fiber and germ from each other and from endosperm;
the steeping stage steeps the endosperm isolated during the fiber and germ separation stage to produce a steeped endosperm;
the protein separation stage separates protein from starch in the steeped endosperm; and
the fermentation stage is adapted to receive a fermentation feed comprising maltodextrin produced in the liquefaction stage and C5-enriched materials produced during pretreatment of a cellulosic feed supply.

48. An ethanol processing plant comprising:
a. a tempering stage;
b. a milling stage;
c. a fiber and germ separation stage;
d. a liquefaction stage;
e. a protein separation stage; and
f. a fermentation stage;
wherein
the tempering stage dries corn to a selected water content;
the fiber and germ separation stage separates fiber and germ from each other and from endosperm;
the liquefaction stage reduces starch in the endosperm to maltodextrin;
the protein separation stage separates protein from maltodextrin produced during the liquefaction stage; and
the fermentation stage is adapted to receive a fermentation feed comprising the maltodextrin and C5-enriched materials produced during pretreatment of a cellulosic feed supply.

49. The method of claim 1, wherein edible corn components are removed, isolated, and/or recovered during the fractionating.

50. The method of claim 32, wherein edible corn components are removed, isolated, and/or recovered prior to cofermenting.
